(19) United States
(12) Patent Application Publication
(10) Pub. No.: US 2008/0161692 A1
(43) Pub. Date: Jul. 3, 2008

(54) Devices and methods for ablation

(76) Inventor: Jonathan L. Podmore, San Carlos, CA (US)

Correspondence Address: SJM/AFD-WILEY, 14901 DEVEAU PLACE, MINNETONKA, MN, 55345-2126, US

(21) Appl. No.: 11/647,306

(22) Filed: Dec. 29, 2006

(51) Int. Cl. A61N 7/00

(52) U.S. Cl. 600/459

(57) ABSTRACT

An ablation device includes at least one ablation cell or element having a piezoelectric layer and the piezoelectric layer includes a surface interrupting feature that alters the ultrasound energy output of the piezoelectric layer compared to a piezoelectric layer of comparable size and shape having no surface interrupting feature. In a second embodiment, at least one surface interrupting feature defines a boundary between multiple segments of a single ablation element such that a lesion having a length less than the full length of an ablation element may be created.

### BACKGROUND OF THE INVENTION

a. Field of the Invention

The instant invention generally relates to devices and methods for treating electrophysiological diseases of the heart. In particular, the instant invention relates to devices and methods for epicardial ablation for the treatment of atrial fibrillation.

b. Background Art

It is well known that atrial fibrillation results from disorganized electrical activity in the heart muscle (the myocardium). Procedures for treating atrial fibrillation may involve the creation of a series of elongated transmural lesions—that is, lesions extending through a sufficient thickness of the myocardium to block electrical conduction—to create conductive corridors of viable tissue bounded by scar tissue. Such procedures may be performed from outside the heart (epicardial ablation) using devices introduced into the patient's chest. Various techniques may be used for the creation of epicardial transmural lesions, including, for example, ultrasound ablation.

In performing epicardial ablations, it is generally considered most efficacious to include a transmural lesion isolating the pulmonary veins from the surrounding myocardium. The pulmonary veins connect the lungs to the left atrium of the heart, joining the left atrial wall on the posterior side of the heart. Epicardial ablation devices and methods useful for creating transmural lesions for the treatment of atrial fibrillation have been described in U.S. Pat. No. 7,052,493 to Vaska et al., which is hereby expressly incorporated by reference as though fully set forth herein. Devices adapted for forming continuous lesions around the pulmonary veins may include a plurality of ablation cells or elements having a focused piezoelectric layer to focus ultrasound energy and may be configured to wrap around the pulmonary veins to deliver high frequency focused ultrasound energy to a tissue. A disadvantage of the current devices is that ultrasound energy emitted from the ends of adjacent ablation cells may overlap, resulting in ultrasound peaks and non-uniform energy distribution across the length of an ablation element or cell. Further, the acoustic waves emitted from an ultrasound transducer tend to rebound off the edges of the transducer resulting in higher intensity at the ends of the transducer and a non-uniform acoustic output.

It is also considered desirable to perform linear ablation at the mitral isthmus, which is defined as extending from the lateral mitral annulus to the ostium of the left inferior pulmonary vein (LIPV). Studies have shown that catheter ablation of the mitral isthmus, in combination with pulmonary vein (PV) isolation, consistently results in demonstrable conduction block and is associated with a high cure rate for paroxysmal atrial fibrillation. Producing precise lesions at these locations is necessary in order to take full advantage of the synergistic benefits of combining linear left atrial ablations, such as the mitral isthmus ablation, with PV isolation. It is important that the lesions have continuity with each other. Failure to provide continuity may allow for reentry pathways, which would limit the effectiveness of the treatment.

In performing linear left atrial ablations in combination with PV isolation, it may be desirable to have discrete control over the length of the lesions that are created. Generally, the length of the lesion correlates to the length of an ablation cell or element. When ablating tissue near structures that it is not desirable to ablate, such as the atrioventricular groove, it may be necessary to create a lesion less than a full length of an ablation element. A disadvantage of existing devices is the inability of such devices to provide individual control over the length of a transmural lesion by allowing for the activation of less than the full length of an ablation cell or element.

### BRIEF SUMMARY OF THE INVENTION

It is desirable to be able to provide an ablation device having a plurality of ablation cells for uniform delivery of ultrasound energy to a tissue.

It is also desirable to provide an ablation device allowing for discrete control over the length of a transmural lesion such that a lesion having a length less than the length of an ablation element or cell may be created.

The present invention meets these and other objectives by providing devices and methods for ablating tissue having shaped or segmented transducers. According to a first embodiment of the invention, a device for ablating tissue includes at least one ultrasound ablation element attached to an elongated body. The at least one ultrasound ablation element includes a piezoelectric layer comprising a piezoelectric material, at least one electrical lead coupled to the piezoelectric layer, and, optionally, a matching layer coupled to the piezoelectric layer. The piezoelectric layer further includes a center region, an outer region and a surface interrupting feature. The surface interrupting feature alters the ultrasound energy output of the piezoelectric layer compared to a piezoelectric layer of similar size a shape having no surface interrupting feature. In one embodiment, the ultrasound energy output is substantially uniform across the surface of the piezoelectric layer. In a second embodiment, the ultrasound energy output of the outer region is less than the ultrasound energy output of the center region. For example, the ultrasound energy output of the outer region may be at least about 5%-50% lower, or about 10% lower, or about 20% lower, or about 30% lower, or about 40% lower, or about 50% lower than the ultrasound energy output of the center region of the piezoelectric layer.

The piezoelectric layer comprises a piezoelectric material such as lead-zirconate-titanate (PZT), a piezoceramic, a piezopolymer material, or a piezocomposite material. The matching layer may comprise a fluorphlogopite mica in a borosilicate glass matrix, aluminum, vitreous carbon, glass or ceramic. In preferred embodiments, the electrical lead is coupled to the center region of the piezoelectric layer. The ablation elements are preferably plano-concave, but may be flat, concave, convex or plano-convex.

The surface interrupting feature may be formed by laser etching the piezoelectric layer. In alternate embodiments, the surface interrupting feature is formed by one or a combination of laser etching, wet etching, dicing, bending, curving or cutting the piezoelectric layer on one surface or, optionally, on both a front and back surface of the piezoelectric layer. The surface interrupting feature may be shaped in the form of an ellipse or may be curvilinear. The width of the surface interrupting feature may be equal to a thickness of the piezoelectric layer or may have a width less than a thickness of the piezoelectric layer. The depth of the surface interrupting feature may be equal to the thickness of the piezoelectric layer, or may have a depth less than a thickness of the piezoelectric layer.

The surface interrupting feature may be formed by electrode shaping wherein one or more metal layers coupled to the piezoelectric layer are cut or etched to remove a portion of the metal, but no portion of the piezoelectric layer is removed. The surface interrupting feature electrically isolates a center region from an outer region such that only the region to which an electrical lead is coupled may be activated to emit ultrasonic energy. Further, electrode shaping and piezoelectric layer shaping may be combined to produce a desired ultrasound energy output. Any combination of surface interrupting features and electrode placement can be used to produce a desired output.

In yet another embodiment, the device includes a plurality of ablation elements, wherein at least one of the plurality of ablation elements includes a surface interrupting feature.

In still another embodiment, a device for ablating tissue includes a shaft having a flexible distal end and at least one ultrasound ablation element coupled to the distal end of the shaft. The ultrasound ablation element includes a piezoelectric layer comprising a piezoelectric material, at least one electrical lead coupled to the piezoelectric layer, and, optionally, a matching layer coupled to the piezoelectric layer. The piezoelectric layer has a center region, an outer region and a surface interrupting feature, and the surface interrupting feature alters the ultrasound energy output of the piezoelectric layer. For example, the surface interrupting feature may cause the ultrasound energy output to be substantially uniform across the length of the piezoelectric layer. Alternatively, the ultrasound energy output of the outer region may be less than the ultrasound energy output of the center region. In a preferred embodiment, the device includes two ablation elements wherein the ablation elements are focused to direct ablating energy at a desired distance from the surface of the elements in contact with a tissue.

A method of producing an ablating device according to the present invention includes providing a piezoelectric layer, shaping the piezoelectric layer to form a surface interrupting feature, wherein the surface interrupting feature separates a center region and an outer region of the piezoelectric layer and measuring the ultrasound output of the piezoelectric layer. The shaping and measuring steps are repeated until a desired ultrasound energy output is obtained. At least one electrical lead is coupled to the center region of the piezoelectric layer, and a matching layer is optionally coupled to the piezoelectric layer. The desired ultrasound energy output is preferably one in which the ultrasound energy output of the outer region is at least about 5%-50% lower, or about 10% lower, or about 20% lower, or about 30% lower, or about 40% lower, or about 50% lower than the ultrasound energy output of the center region of the piezoelectric layer. Alternatively, the desired ultrasound energy output is substantially uniform across the surface of the piezoelectric layer.

The shaping step may include at least one of laser etching, wet etching, dicing, bending, curving or cutting the piezoelectric layer. The matching layer is preferably acoustically coupled to the piezoelectric layer. The present invention also includes a transducer made according to the foregoing method and incorporated into an ablation device.

In another aspect of the method of producing an ablating device having a surface interrupting feature, the surface interrupting feature can be prepared by electrically isolating separate regions of the piezoelectric element. In effect, only certain regions of the piezoelectric surface will be activated by the electrical lead to output ultrasound energy. The electrical isolating and shaping aspects can both be performed in producing a single ablating element.

In yet another embodiment, the invention relates to a device for ablating tissue having at least one ultrasound ablation element, the at least one ultrasound ablation element having a piezoelectric layer having multiple segments. A surface interrupting feature separates a first segment and a second segment of the piezoelectric layer and at least one electrical lead is coupled to each of the first and second segments such that the segments may be separately activated. In further embodiments, the piezoelectric layer includes three or four separately activatable segments.

A method of ablating tissue according to the present invention includes providing an ablating device having at least one ultrasound ablation element, the at least one ultrasound ablation element comprising a piezoelectric layer having at least two separately activatable segments, manipulating the ablation device about an epicardial surface such that the at least one ablation element is positioned over tissue to be ablated, and ablating tissue by activating at least one of the separately activatable segments.

A method of manufacturing an ablating device according to the present invention includes providing a piezoelectric layer, shaping the piezoelectric layer to form a first surface interrupting feature, the first surface interrupting feature forming a boundary between a first segment and a second segment, coupling at least one electrical lead to each of the first and second segments of the piezoelectric layer, and, optionally, coupling a matching layer to the piezoelectric layer. The method may further include shaping the piezoelectric layer to form additional surface interrupting features to create additional segments that are separately activatable.

The foregoing and other aspects, features, details, utilities, and advantages of the present invention will be apparent from reading the following description and claims, and from reviewing the accompanying drawings.

### BRIEF DESCRIPTION OF THE DRAWINGS

FIG. 1 illustrates an ablation device according to one embodiment of the present invention.

FIG. 2 depicts an ablation element within a housing.

FIG. 3 illustrates a flat ablation element.

FIG. 4 depicts a concave ablation element.

FIG. 5 illustrates a convex ablation element.

FIG. 6 depicts a saddle-shaped ablation element.

FIG. 7 illustrates a plano-concave ablation element

FIG. 8 depicts a plano-convex ablation element.

FIG. 9 illustrates a top view of an ablation element having an elliptical-shaped surface interrupting feature.

FIG. 10 illustrates a top view of an ablation element having a curvilinear surface interrupting feature.

FIG. 11 illustrates a top view of an ablation element having two active segments.

FIG. 12 depicts a top view of an ablation element having three active segments.

FIG. 13 illustrates a top view of an ablation element having four active segments.

FIG. 14 depicts another ablation device according to the instant invention.

### DETAILED DESCRIPTION OF THE INVENTION

As used herein, the words “preferred,” “preferentially,” and “preferably” refer to embodiments of the invention that afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the invention and no disclaimer of other embodiments should be inferred from the discussion of a preferred embodiment or a figure showing a preferred embodiment.

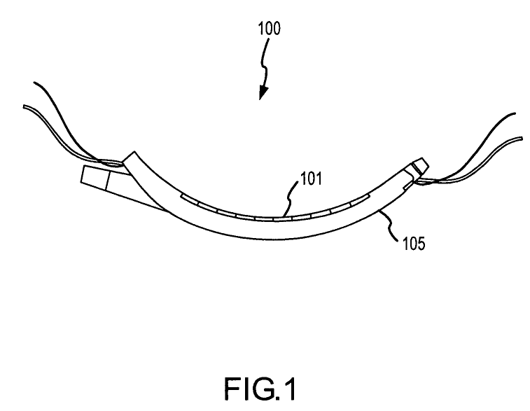

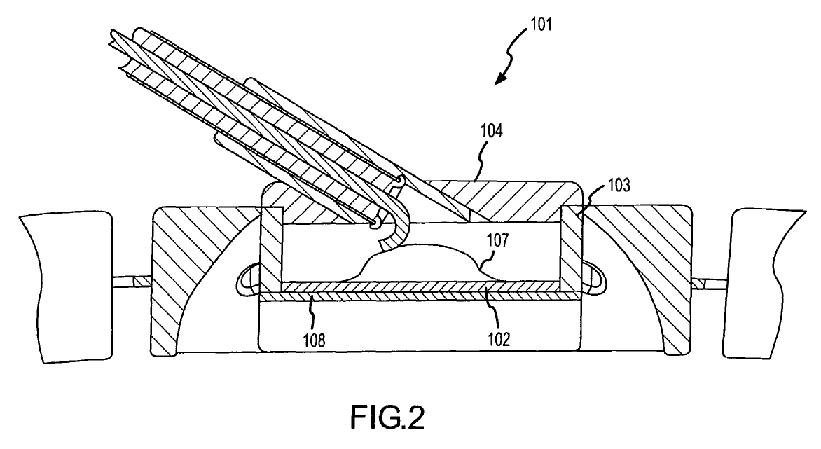

Referring to FIGS. 1-3, an ablation device 100 according to one embodiment of the present invention is shown. The ablation device 100 includes a plurality of ablation elements 101 coupled to an elongated body 105. Body 105 may have a curved surface. Ablation elements 101 may be substantially aligned, meaning there is little or no staggering between ablation elements 101 along the direction in which they are coupled together.

Each ablation element 101 includes a piezoelectric layer 102 comprising a piezoelectric material. Piezoelectric layer 102 may be secured within a housing. The housing includes side walls 103 and a top 104. In preferred embodiments, a matching layer 108 is coupled to piezoelectric layer 102; however, a matching layer is not required. Matching layer 108 may be bonded or otherwise acoustically coupled to piezoelectric layer 102. An electrical lead 107 is also coupled to piezoelectric layer 102. The electrical lead 107 is preferably a copper ribbon; however, a person of skill in the art will appreciate that any suitable type of electrical lead may be used without departing from the spirit and scope of the invention.

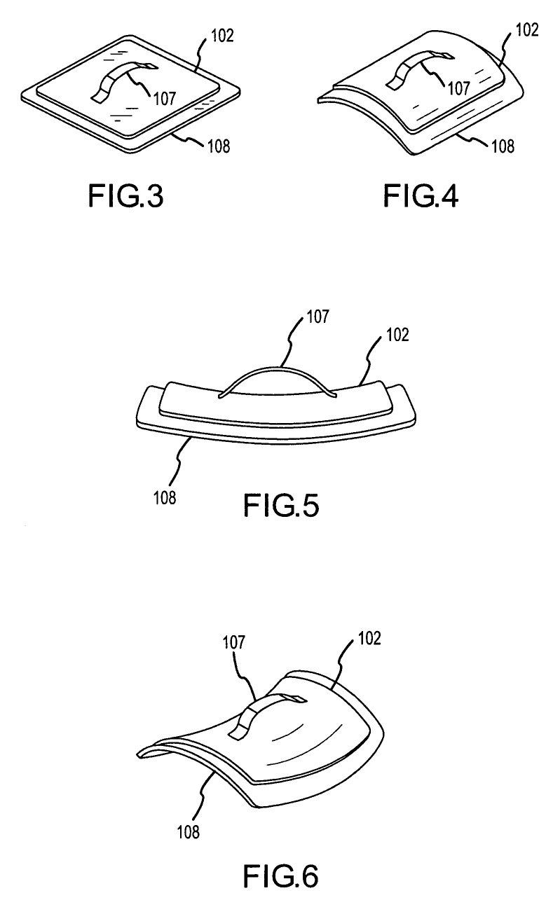

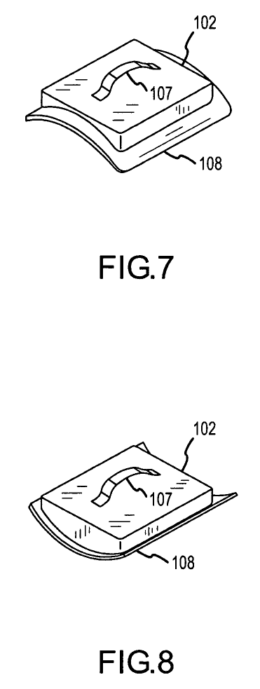

Ablation elements 101 preferably have a width of about 1 mm to about 15 mm, and more preferably of about 10 mm, and a length of about 2 mm to about 25 mm, and more preferably of about 12 mm. In preferred embodiments, piezoelectric layer 102 is plano-concave, as shown in FIG. 7, and delivers focused ultrasound energy that is focused in at least one direction. In alternative embodiments, piezoelectric layer 102 may be substantially flat (see FIG. 3), concave (see FIG. 4), convex (see FIG. 5), saddle-shaped (see FIG. 6), or plano-convex (see FIG. 8).

Device 100 preferably has from about 5 to about 30 ablation elements, more preferably from about 10 to about 25 ablation elements, and most preferably less than about 15 ablation elements. It should be understood, however, that any number of ablation elements 101 may be used depending upon the specific application for ablation device 100. For example, ablation device 100 may be used to extend around multiple vessels, such as the four pulmonary veins, or around only a single vessel, such as the aorta, a pulmonary vein, the superior vena cava, or inferior vena cava, in which case ablation device 100 preferably includes about 4 to about 12 ablation elements, and more preferably includes about 8 ablation elements.

Piezoelectric layer 102 preferably comprises lead-zirconate-titanate (PZT), but may comprise any piezoelectric material, for example barium titanate, a piezoceramic, a piezopolymer material, or a piezocomposite material. In preferred embodiments, matching layer 108 comprises a fluorphlogopite mica in a borosilicate glass matrix, such as Macor®. Matching layer may alternatively comprise aluminum, aluminum nitride, boron nitride, silicon nitride, graphite, vitreous carbon, silicon carbide, cermets, glasses coated with thermally conductive films, or any combinations thereof. Matching layer 108 is positioned between piezoelectric layer 102 and a tissue to be ablated. Matching layer 108 minimizes acoustic reflections, enhances spectral performance and more efficiently transmits acoustic energy into a patient's body.

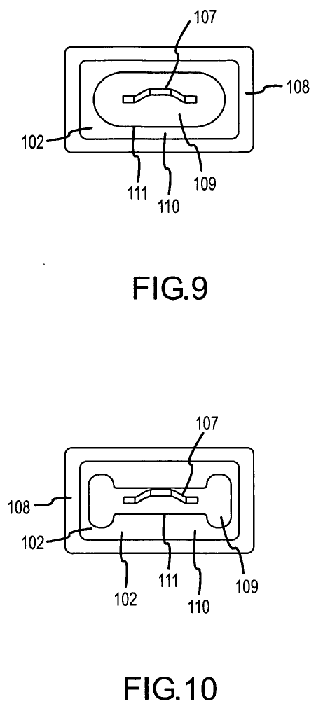

Referring now to FIGS. 9-10, piezoelectric layer 102 has a center region 109 and an outer region 110. Center region 109 is separated from outer region 110 by a surface interrupting feature 111. Surface interrupting feature 111 is a region of piezoelectric layer 102 that causes a change in the acoustic output across the surface of piezoelectric layer 102. In one embodiment, the surface interrupting feature causes an ultrasound energy output that is substantially uniform across the surface of the piezoelectric layer. When a surface interrupting feature is not present, the acoustic output may peak on the ends of the piezoelectric layer due to the rebound of the ultrasound waves against the edges of the piezoelectric layer. The surface interrupting feature can be shaped to eliminate the output peaks and create a substantially uniform acoustic output. By “substantially uniform” it is meant that the acoustic output across the surface of the piezoelectric element does not vary by more than about 5%, or by not more than about 20%.

In an alternative embodiment, the surface interrupting feature 111 is shaped such that the ultrasound output is greatest at center region 109 of piezoelectric layer 102 and becomes more attenuated near outer region 110. When adjacent ablation elements are activated, the combined energy delivered from the overlapping outer regions will be substantially equal to the ultrasound energy delivered from the center region of each element. Preferably the ultrasound energy output from outer region 110 is reduced by at least about 10%-80% relative to the ultrasound energy output from center region 109, more preferably at least about 30%-70% relative to the ultrasound energy output from center region 109, and most preferably at least about 40%-60% relative to the ultrasound energy output from center region 109. However, the ultrasound energy output from outer region 110 can be less than about 10% or more than about 80% relative to the ultrasound energy output from center region 109 without departing from the spirit and scope of the present invention.

Surface interrupting feature 111 may be, for example, a groove, cut or etching. In preferred embodiments, surface interrupting feature 111 is formed by laser-etching piezoelectric layer 102 to remove a portion of the piezoelectric material. Both a front and back surface of the piezoelectric layer may be etched, or alternatively, only one surface of the piezoelectric layer may be etched. The etched portion of piezoelectric layer 102 may be a thin strip in the shape of a circle or ellipse enclosing center region 109 of piezoelectric layer 102 as shown in FIG. 9. Alternatively, surface interrupting feature 111 may be curvilinear or bone-shaped, as shown in FIG. 10. A person of skill in the art will appreciate, however, that surface interrupting feature 111 can take any suitable shape that produces a desired ultrasound energy output. For example, surface interrupting feature 111 may be shaped in the form of a partial circle or partial ellipse.

Surface interrupting feature 111 preferably has a width equal to a thickness of piezoelectric layer 102, but surface interrupting feature 111 may have a width greater than or less than a thickness of piezoelectric layer 102. Further, surface interrupting feature 111 may have a depth equal to a thickness of piezoelectric layer 102, or may have a depth less than a thickness of piezoelectric layer 102. For example, if the surface interrupting feature is formed by cutting or laser-etching, the cut or etching may extend through the entire thickness of piezoelectric layer 102, or it may extend only through a portion of the thickness.

To produce an ultrasound transducer having a desired ultrasound energy output, the transducer may be tuned. To tune an ultrasound transducer, piezoelectric layer 102 is shaped to form a surface interrupting feature 111, for example using laser etching techniques, and the acoustic output is measured. The acoustic output may be measured using known techniques and instruments, such as, for example, a hydrophone. The shaping and measuring steps are repeated until a desired acoustic output is obtained. For example, a small elliptical etching may be created at a certain depth on a piezoelectric layer 102 and the energy output may be measured. Then a second small etching may be created on piezoelectric layer 102 at the same or a different depth as the first etching, and the energy output may be measured again. The desired ultrasound energy output is preferably one in which the ultrasound energy emitted from outer region 110 is less than the ultrasound energy emitted from center region 109, but may also be one in which the ultrasound energy output is uniform across the piezoelectric layer.

While a surface interrupting feature formed by laser etching has been described, other methods of producing a surface interrupting feature are within the spirit and scope of the present invention. A person of skill in the art will appreciate that any method may be used to create a surface interrupting feature that alters the ultrasound energy output of piezoelectric layer 102. For example, surface interrupting feature 111 may be created by wet etching piezoelectric layer 102 or by dicing, bending, curving or cutting piezoelectric layer 102.

In preferred embodiments, an electrical lead 107 is coupled to center region 109 of piezoelectric layer 102, but more than one electrode may be used without departing from the spirit and scope of the invention. Electrical lead 107 is also connected to a power source and provides power that drives piezoelectric layer 102. When powered, center region 109 becomes activated and delivers ultrasound energy to a tissue. The surface interrupting feature electrically isolates the outer region 110 from the center region 109 to alter the ultrasound energy output of the piezoelectric element 101.

Piezoelectric layer 102 may be coated or plated with one or more metal layers to form an electrode layer, and electrical lead 107 may be coupled to the electrode layer. In preferred embodiments, surface interrupting feature 111 is formed by electrode shaping wherein portions of the one or more metal layers are removed, for example by cutting or etching, but no portion of the piezoelectric layer is removed. In other words, only the electrode layer is shaped to form a surface interrupting feature. As previously described herein, the surface interrupting may be in the shape of an ellipse, for example, and may form the boundary between an outer region and a center region of the electrode layer. The surface interrupting feature electrically isolates the outer region from the center region such that only the region to which an electrical lead is coupled may be activated. The electrode layer preferably comprises one layer of nickel and a second layer of gold; however a person of skill in the art will appreciate that any suitable metal or combination of metals can be used without departing from the spirit and scope of the invention. For example, other metals that may be used include, silver, copper and platinum. The electrode layer preferably has a thickness of about 1000 Angstroms, but may have a thickness of about 500 Angstroms to about 5000 Angstroms. The electrode layer may be coupled to a front or back side of a piezoelectric layer or may be coupled to both a front and back side of a piezoelectric layer.

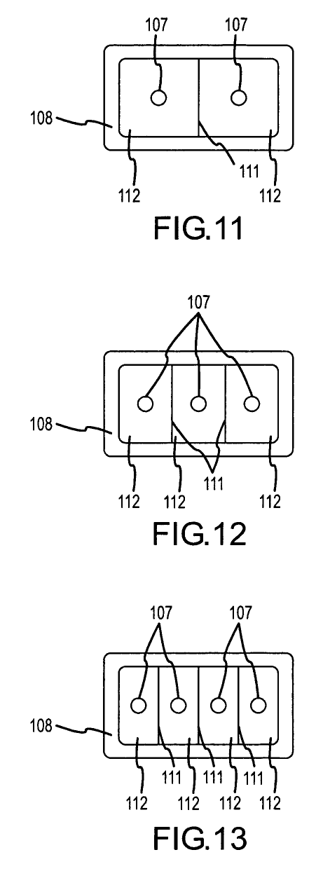

Referring to FIGS. 11-13, in another embodiment, surface interrupting feature 111 may be shaped to form a substantially straight line that divides piezoelectric layer 102 into distinct segments 112. A single surface interrupting feature may be used to divide piezoelectric layer 102 into two distinct segments 112, as shown in FIG. 11. Alternatively, two surface interrupting features 111 may be used to divide piezoelectric layer 102 into three distinct segments 112 (see FIG. 12), or three surface interrupting features 111 may be used to divide piezoelectric layer 102 into four distinct segments 112 (see FIG. 13). A person of skill in the art will appreciate that additional surface interrupting features may be used to divide piezoelectric layer 102 into larger numbers of segments 112.

At least one electrical lead 107 is coupled to at least one of the segments 112 of piezoelectric layer 102, and each segment 112 may be separately activatable. By separately activatable it is meant that a user can selectively cause one or more of the segments to deliver ultrasound energy to a tissue. For example, a first segment of piezoelectric layer 102 may be activated while a second segment remains inactive. Further, for a piezoelectric layer 102 having three segments, the first two segments may be activated while the third segment is inactive, or the first and third segments may be activated while the second segment remains inactive. The separately activatable segments allow for greater control of the ablation by providing a mechanism for activating only a fraction of an ablation element. In this manner, a lesion less than the full length of an ablation element may be created.

In preferred embodiments, a surface interrupting feature is formed on an electrode layer coupled to a piezoelectric layer and divides an electrode layer into distinct segments. The surface interrupting feature electrically isolates each segment from the other segments. At least one electrical lead is coupled to each segment such that each segment may be independently activated.

Although it is preferred to vary the frequency of the energy delivered to the ablation elements 101 when ablating the tissue, the ablation elements may, of course, be operated at a single frequency. Various treatment methods for delivering energy to the ablation elements are described in U.S. Pat. No. 7,052,493. In a first treatment method, the ablation elements are activated at a frequency of about 2 MHz to about 7 MHz, and preferably of about 3.5 MHz, and a power of about 80 watts to about 150 watts, and preferably of about 130 watts, in short bursts. Following treatment at the first frequency, the ablation elements are preferably operated at a frequency of about 2 MHz to about 14 MHz, more preferably about 3 MHz to about 7 MHz, and most preferably about 6 MHz, and a power of about 20 watts to about 80 watts, and preferably about 60 watts. As a final treatment, the ablation elements are preferably operated at a frequency of at least about 3 MHz and about 16 MHz, and preferably at about 6 MHz. In a preferred method, the ablation elements are operated at about 2 watts to about 20 watts, and more preferably about 15 watts.

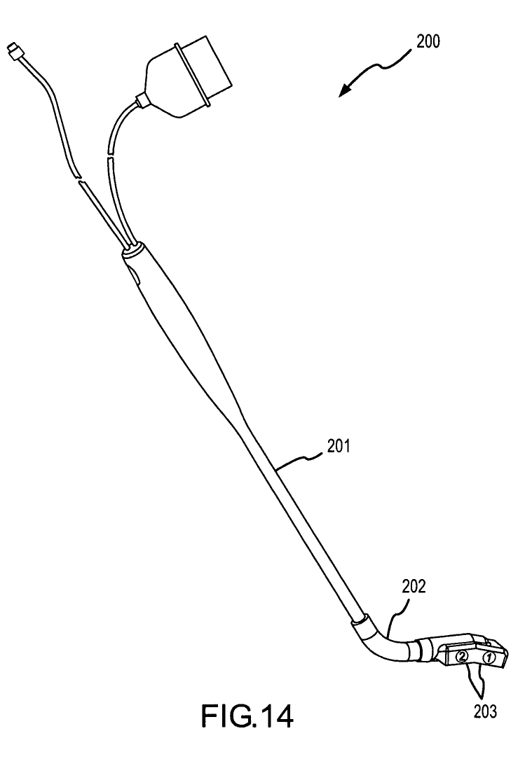

Referring now to FIG. 14, an ablation device according to another embodiment of the present invention is shown. The device 200 has a shaft 201, which is relatively rigid, with a flexible distal portion 202. The distal portion 202 of the shaft 201 can be shaped by a user (i.e., a physician) into a variety of positions to accommodate the angle of introduction of the ablating cells 203 into the patient and the target surface orientation. The distal portion may include a stacked coil contained within a sheath that can be deformed by the user and retain the deformed shape.

In a particularly preferred device as shown in FIG. 14, the device 200 has at least one ablation element, and preferably two ablation cells or elements 203. The ablation device may, of course, have more than two ablation elements. The ablation elements may be fixed relative to one another, or, alternatively, may have a flexible or malleable connection therebetween in order to adjust the relative orientation or position of ablation elements.

The ablation elements 203 may have all of the features of the ablation elements previously described with respected to the ablation device shown in FIG. 1, including a piezoelectric layer 102, a matching layer 108, and an electrical lead 107. Piezoelectric layer 102 may further include a center region 109, an outer region 110, and at least one surface interrupting feature 111 separating center region 109 and outer region 110, or defining distinct segments 112 of piezoelectric layer 102.

Although several embodiments of this invention have been described above with a certain degree of particularity, those skilled in the art could make numerous alterations to the disclosed embodiments without departing from the spirit or scope of this invention. All directional references (e.g., upper, lower, upward, downward, left, right, leftward, rightward, top, bottom, above, below, vertical, horizontal, clockwise, and counterclockwise) are only used for identification purposes to aid the reader's understanding of the present invention, and do not create limitations, particularly as to the position, orientation, or use of the invention. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other. It is intended that all matter contained in the above description or shown in the accompanying drawings shall be interpreted as illustrative only and not limiting. Changes in detail or structure may be made without departing from the spirit of the invention as defined in the appended claims.

What is claimed is:

1. A device for ablating tissue, comprising:
at least one ultrasound ablation element coupled to an elongated body, the at least one ultrasound ablation element having
a piezoelectric layer comprising a piezoelectric material; and
at least one electrical lead coupled to the piezoelectric layer,
wherein the piezoelectric layer has a center region, an outer region and a surface interrupting feature, and wherein the surface interrupting feature alters the ultrasound energy output of the piezoelectric layer.

2. The device of claim 1, wherein the at least one ultrasound ablation element further comprises a matching layer coupled to the piezoelectric layer.

3. The device of claim 2, wherein the ultrasound energy output of the outer region is less than the ultrasound energy output of the center region.

4. The device of claim 3, wherein the ultrasound energy output of the outer region is at least about 10% less than the ultrasound energy output of the center region.

5. The device of claim 2, wherein the ultrasound energy output is substantially uniform across the piezoelectric layer.

6. The device of claim 2, wherein the surface interrupting feature is formed by laser etching the piezoelectric layer.

7. The device of claim 2, wherein the surface interrupting feature is formed by at least one of laser etching, wet etching, dicing, bending, curving or cutting the piezoelectric layer.

8. The device of claim 2, wherein the at least one electrical lead is coupled to the center region of the piezoelectric layer.

9. The device of claim 2, wherein the surface interrupting feature is shaped in the form of an ellipse.

10. The device of claim 2, wherein the surface interrupting feature is curvilinear.

11. The device of claim 2, wherein a width of the surface interrupting feature is equal to a thickness of the piezoelectric layer.

12. The device of claim 2, wherein a width of the surface interrupting feature is less than a thickness of the piezoelectric layer.

13. The device of claim 1, having a plurality of ablation elements, wherein at least one of the plurality of ablation elements comprises a surface interrupting feature.

14. The device of claim 1, wherein the piezoelectric material is selected from the group consisting of lead-zirconate-titanate (PZT), a piezoceramic, a piezopolymer material, or a piezocomposite material.

15. The device of claim 2, wherein the matching layer is selected from fluorphlogopite mica in a borosilicate glass matrix, aluminum, aluminum nitride, boron nitride, silicon nitride, graphite, vitreous carbon, silicon carbide and cermets.

16. The device of claim 2, wherein the at least one ultrasound ablation element is plano-concave and is configured to emit focused ultrasound energy that is focused in at least one direction.

17. The device of claim 2, having a plurality of substantially aligned ultrasound ablation elements.

18. The device of claim 2, having a shaft with a flexible distal end and at least one ultrasound ablation element coupled to the distal end of the shaft.

19. A device for ablating tissue, comprising:
at least one ultrasound ablation element coupled to an elongated body, the at least one ultrasound ablation element having
a piezoelectric layer comprising a piezoelectric material;
an electrode layer coupled to the piezoelectric material; and
at least one electrical lead coupled to the electrode layer,
wherein the electrode layer has a center region, an outer region and a surface interrupting feature, and wherein the surface interrupting feature electrically isolates the outer region from the center region.

20. The device of claim 19, wherein the at least one ultrasound ablation element further comprises a matching layer coupled to the electrode layer.

21. The device of claim 19, wherein the at least one electrical lead is coupled to the center region of the electrode layer.

22. The device of claim 19, wherein the surface interrupting feature is formed by laser etching the electrode layer.

23. The device of claim 19, wherein the surface interrupting feature is formed by at least one of laser etching, wet etching, dicing, bending, curving or cutting the electrode layer.

24. The device of claim 19, wherein the electrode layer comprises at least one of gold, nickel, silver, copper and platinum.

25. A method of ablating cardiac tissue from an epicardial location, comprising:
providing an ablating device having at least one ultrasound ablation element, the at least one ultrasound ablation element having a piezoelectric layer comprising a center region, an outer region and a surface interrupting feature, wherein the surface interrupting feature alters the ultrasound energy output of the piezoelectric layer;
manipulating the ablation device about an epicardial surface such that the at least one ultrasound ablation element is positioned over tissue to be ablated; and
ablating tissue by activating the at least one ultrasound ablation element.

26. A method of producing an ultrasound ablating device, comprising:
providing a piezoelectric layer;
shaping the piezoelectric layer to form a surface interrupting feature, wherein the surface interrupting feature separates a center region and an outer region of the piezoelectric layer;
measuring ultrasound output of the piezoelectric layer;
repeating the shaping and measuring steps until a desired ultrasound energy output is obtained; and
coupling at least one electrical lead to the center region of the piezoelectric layer.

27. The method of claim 26, further comprising coupling a matching layer to the piezoelectric layer.

28. The method of claim 27, wherein the matching layer is selected from fluorphlogopite mica in a borosilicate glass matrix, aluminum, aluminum nitride, boron nitride, silicon nitride, graphite, vitreous carbon, silicon carbide and cermets.

29. The method of claim 26, wherein the desired ultrasound energy output is one in which the ultrasound energy output of the outer region is at least about 10% less than the ultrasound energy output of the center region.

30. The method of claim 26, wherein the desired ultrasound energy output is substantially uniform across the piezoelectric layer.

31. The method of claim 26, wherein the piezoelectric material is selected from the group consisting of lead-zirconate-titanate (PZT), a piezoceramic, a piezopolymer material, or a piezocomposite material.

32. The method of claim 26, wherein the shaping step comprises laser etching the piezoelectric layer.

33. The method of claim 26, wherein the shaping step comprises at least one of laser etching, wet etching, dicing, bending, curving or cutting the piezoelectric layer.

34. The method of claim 26, wherein the surface interrupting feature is shaped in the form of an ellipse.

35. The method of claim 26, wherein the surface interrupting feature is curvilinear.

36. A transducer made according to the method of claim 26, the transducer being incorporated into an ablation device.

37. A device for ablating tissue, comprising:
at least one ultrasound ablation element, the at least one ultrasound ablation element having
a piezoelectric layer having a first segment, a second segment and a first surface interrupting feature separating the first and second segments; and
at least one electrical lead coupled to each of the first and second segments of the piezoelectric layer,
wherein the first and second segments are separately activatable.

38. The device of claim 37, further comprising a matching layer coupled to the piezoelectric layer.

39. The device of claim 37, wherein the first surface interrupting feature is formed by laser etching the piezoelectric layer.

40. The device of claim 37, wherein a width of the first surface interrupting feature is equal to a thickness of the piezoelectric layer.

41. The device of claim 37, wherein a width of the first surface interrupting feature is less then a thickness of the piezoelectric layer.

42. The device of claim 37, wherein the piezoelectric layer further comprises a third segment and a second surface interrupting feature, wherein the second surface interrupting feature separates the second and third segments, and wherein the first, second and third segments are separately activatable.

43. The device of claim 42, wherein the piezoelectric layer further comprises a fourth segment and a third surface interrupting feature, wherein the third surface interrupting feature separates the third and fourth segments, and wherein the first, second, third and fourth segments are separately activatable.

44. A method of ablating cardiac tissue from an epicardial location, comprising:
providing an ablating device having at least one ultrasound ablation element, the at least one ultrasound ablation element comprising a piezoelectric layer having at least two segments and at least one surface interrupting feature, wherein each segment is separately activatable;
manipulating the ablation device about an epicardial surface such that the at least one ablation element is positioned over tissue to be ablated; and
ablating tissue by activating at least one of the segments of the at least one ablation element.

45. A method of producing an ablating device, comprising:
providing a piezoelectric layer;
shaping the piezoelectric layer to form a first surface interrupting feature, the first surface interrupting feature forming a boundary between a first segment and a second segment;
coupling a matching layer to the piezoelectric layer; and
coupling at least one electrical lead to each of the first and second segments of the piezoelectric layer.

46. The method of claim 45, wherein the shaping step comprises at least one of laser etching, wet etching, dicing, bending, curving or cutting the piezoelectric layer.

47. The method of claim 45, further comprising the step of shaping the piezoelectric layer to form a second surface interrupting feature, the second surface interrupting feature forming the boundary between the second segment and a third segment of the piezoelectric layer.

48. The method of claim 47, further comprising the step of shaping the piezoelectric layer to form a third surface interrupting feature, the third surface interrupting feature forming the boundary between the third segment and a fourth segment of the piezoelectric layer.
